(19) United States
(12) Patent Application Publication
(10) Pub. No.: US 2003/0105165 A1
(43) Pub. Date: Jun. 5, 2003

(54) Gap junctions and EDHF

(76) Inventor: Tudor Morley Griffith, Cardiff (GB)

Correspondence Address: YOUNG & THOMPSON, 745 SOUTH 23RD STREET 2ND FLOOR, ARLINGTON, VA, 22202

(21) Appl. No.: 10/270,663

(22) Filed: Oct. 16, 2002

Related U.S. Application Data
(60) Provisional application No. 60/330,776, filed on Oct. 31, 2001.

(30) Foreign Application Priority Data
Oct. 17, 2001 (GB) 0125055.4
Oct. 17, 2001 (GB) 0125040.6

(51) Int. Cl. A61K 31/137

(52) U.S. Cl. 514/651

(57) ABSTRACT

The invention relates to the permeability of gap junctions and specifically to agents which modulate same. The invention describes the use of cAMP and/or cAMP phosphodiesterase inhibitors to enhance flow through gap junctions and various synthetic peptides which attenuate flow through gap junctions.

This invention relates to the use of cyclic adenosine monophosphate (cAMP), an adenylyl cylase activator, or cAMP phosphodiesterase (PDE) inhibitors, or pharmaceutically acceptable derivatives and salts thereof, in the treatment of disease or disorders in animals, including man, which respond to modulation of the Endothelium-derived hyperpolarising factor (EDHF), and pharmaceutical preparations containing such cAMP, adenylyl cylase activators or cAMP PDE inhibitors. The invention also extends to synthetic peptides capable of inhibiting or attenuating intercellular gap junction communication both per se and for use in production of pharmaceutical compositions. The invention also extends to the use of cAMP, adenylyl cylase activators or CAMP PDE inhibitors in combination with a therapeutic substance to assist or enhance a transfer of the substance from the application region into the subjacent cells. Still further the invention extends to a pharmaceutical composition in which a therapeutic substance is linked to a moiety designed to render the therapeutic substance permeant to a cell membrane whereafter the moiety is cleaved from the therapeutic substance to allow it to pass into the subjacent tissue via one or more intercellular gap junctions.

If arteries become narrow or dysfunctional there may be reductions in the supply of oxygen and nutrients to the major organs. Most notably, in the heart this may cause angina or infarction, and in the brain this may cause stroke. Research into the mechanisms that regulate arteries is an essential step in the prevention of vascular disease, as only a full understanding of normal physiology will enable us to develop new therapeutic strategies.

Arteries consist of tubes of muscle lined with a single layer of endothelial cells. The muscle layer possesses the ability to contract and relax, as in other types of muscle. Importantly, it has become apparent that the endothelium synthesises potent chemicals that promote muscle relaxation and thereby increase blood flow. One such substance is the gas nitric oxide which diffuses freely into the vessel wall after being released by the endothelium. Much interest has focused on this mechanism in recent years. Another pathway that can lead to the relaxation of muscle cells in the vessel wall involves changes in the electrical potential of their cell membrane. It has been hypothesised that such changes can be mediated by a so-called endothelium derived hyperpolarising factor or EDHF. Indeed, EDHF-type responses are prominent in small vessels and may be particularly important in diseased states were NO activity is depressed as there is a reciprocal interaction between the two pathways (4,17).

In marked contrast to nitric oxide, our work suggests that an EDHF may transfer preferentially from the endothelium to the muscle by direct cell to cell communication via gap junctions rather than the extracellular space. Gap junctions are membrane structures constructed from connexin (Cx) proteins that cross the cell membrane to dock and form a pore between coupled cells that allows the passage of electrical current and small signalling molecules. Three main subtypes of connexin are present in endothelial and arterial muscle cells (Cxs 37, 40 and 43, classified according to molecular weight) with clusters of up to several hundred individual gap junctions being distributed in plaques at points of cell contact. We have developed a variety of synthetic peptides (33) that can be targeted against specific connexin subtypes and have shown that such peptides can block relaxations conventionally attributed to EDHF. It remains to be established conclusively whether the signal that passes through gap junctions coupling endothelial and muscle cells is electrical or chemical in nature. Nevertheless, our work suggests that the EDHF response is associated with the formation of a chemical signalling messenger, known as cyclic AMP, within muscle cells (27). This closely parallels the action of nitric oxide which activates the synthesis a similar chemical messenger called cyclic GMP.

We have compared the mechanisms that contribute to EDHF-type relaxations evoked by acetylcholine (ACh) and the Ca²⁺ ionophore A23187 in rabbit iliac artery. Relaxations to both agents were associated with ¹⁸1.5-fold elevations in smooth muscle cAMP levels and were attenuated by the adenylyl cyclase inhibitor 2′,5′-dideoxyadenosine (2′,5′-DDA) and potentiated by the cAMP phosphodiesterase inhibitor 3-isobutyl-1-methylxanthine (IBMX). Mechanical responses were inhibited by coadministration of the K_{Ca} channel blockers apamin and charybdotoxin, both in the absence and presence of IBMX, but were unaffected by blockade of K_{ATP} channels with glibenclamide. Relaxations and elevations in cAMP evoked by ACh were abolished by 18α-glycyrrhetinic acid which disrupts gap junction plaques, whereas responses to A23187 were unaffected by this agent. Consistently, in “sandwich” bioassay experiments A23187, but not ACh, elicited extracellular release of a factor that evoked relaxations that were inhibited by 2′,5′-DDA and potentiated by IBMX. We conclude that EDHF-type relaxations of rabbit iliac arteries evoked by ACh and A23187 depend on cAMP accumulation in smooth muscle, but involve signalling via myoendothelial gap junctions and the extracellular space, respectively.

Our finding, that cAMP affects the permeability of gap junctions is clearly of importance and, in so far as the invention is concerned, we believe the finding is particularly, but not exclusively, important in blood vessels, the skin and the bronchial tree. Clearly, where pharmaceuticals are employed to treat diseases and disorders of these tissues then any pharmaceutical product that needs to penetrate the cellular layers and particularly those products that pass through gap junctions will be facilitated in this penetration by the presence of cAMP. This will be especially true of pharmaceutical products that either directly or indirectly, as a primary or secondary consequence of their penetration or activity, attenuate levels of cyclic AMP. A good example of such a product would be an antiviral product. There exists a wide range of antiviral products which are typically nucleoside analogues. Some of these analogues inhibit the enzyme adenylyl cyclase and so reduce levels of cAMP. This realisation was serendipitous because although it is common to use nucleoside analogues as antiviral agents it is not common to think of these agents as inhibitors of cyclic AMP. That is to say, individuals working in the viral field view nucleoside analogues as agents that inhibit viral reverse transcriptase via chain termination. Similarly, cell biologists looking at cell signalling mechanisms are aware that dideoxyadenosine (DDA) can be used to inhibit adenylyl cyclase, but workers in this field do not prescribe an antiviral activity to this agent. It was therefore quite by chance that our work led us into an area where the two fields overlapped and we therefore came to realise that an antiviral agent, when initially administered, via its ability to reduce cAMP actually impedes its own penetration into target tissue by causing the closure of gap junctions. It therefore follows that this ‘auto-impedance’ could be corrected by the co-administration of cAMP and/or adenylyl cylase and/or a cAMP phosphodiesterase inhibitor.

### SUMMARY OF THE INVENTION

According to one aspect of this invention there is provided the use of cAMP (cyclic adenosine monophosphate), or an adenylyl cylase activator, or a cAMP PDE (phosphodiesterase) inhibitor, or a pharmaceutically acceptable derivative or salt thereof, in the production of a pharmaceutical composition effective for the curative or prophylactic treatment of a condition, disease or disorder in an animal, including man, which is responsive to modulation of Endothelium-derived hyperpolarising factor (EDHF).

According to a further aspect of this invention there is provided the use of cAMP (cyclic adenosine monophosphate) PDE (phosphodiesterase) inhibitor, or a pharmaceutically acceptable derivative or salt thereof, in the production of a pharmaceutical composition effective for the curative or prophylactic treatment of a condition, disease or disorder in an animal, including man, which is responsive to modulation of Endothelium-derived hyperpolarising factor (EDHF).

The adenylyl cylase activator may typically be an exogenous or synthetic activator such as salbutamol.

The PDE inhibitor may typically be an exogenous or synthetic cAMP PDE inhibitor such as IBMX (isobutyl-methylxanthine), Rolipram and Milrinone, although this list is not exhaustive. Alternatively, the cAMP PDE inhibitor may be a suitable isolated endogenous cAMP PDE inhibitor.

In a preferred aspect of the invention said use of cAMP, an adenylyl cylase ativator, or a cAMP phosphodiesterase inhibitor is in the production of an antiviral composition. In its simplest form, this aspect of the invention comprises cAMP and/or an adenylyl cylase activator and/or a cAMP phosphodiesterase inhibitor in combination with an antiviral agent. The combination may comprise the cojoining or colinking of the constituents of the composition or, alternatively, simply their copresentation, ideally at pharmacologically active concentrations, within the composition. The following is an inexhaustive list of antiviral agents that may be suitably employed in the aforementioned composition: dideoxyadenosine, zidovudine, didanosine, zalcitabine, stavudine, lamivudine, ganciclovir, foscarnet, cidofovir, lodenosine, acyclovir, or indeed any pharmacologically effective analogues thereof.

Exemplified herein is the antiviral agent 2′,3′-dideoxyadenoine. This lipophilic antiviral agent is converted into a polar triphosphate once it has crossed the cell membrane and in its polar state it is able to penetrate into subcellular layers in its active form. More preferably, more lipophilic or hydrophobic derivatives of antiviral agents may be used. They are commonly known to those skilled in the art and some are described in detail in reference 32.

Our work, to be described below, shows for the first time that cAMP levels increase in smooth muscle cells during the EDHF response. We believe that a diffusible factor may be involved in mediating this biochemical response.

Our work has also demonstrated that the EDHF response may be modulated or attenuated by blocking or inhibiting the intercellular gap junctions. Furthermore there is a wide range of diseases or disorders that respond to inhibition or attenuation of intercellular gap junction communication.

Accordingly, in a further aspect of this invention there is provided use of one or more synthetic peptides homologous to respective portions of one or more connexin proteins and effective to inhibit or attenuate intercellular gap junction communication, in the production of a pharmaceutical composition effective for the curative or prophylactic treatment of a condition, disease or disorder in an animal, including man, that is responsive to inhibition or attenuation of intercellular gap junction communication.

The cAMP, adenylyl cylase activator or cAMP PDE inhibitors and synthetic peptides may be used in the treatment or in the preparation of compositions for the treatment of a wide range of diseases or disorders, for example:—

disease or disorder of the vascular system;

disease or disorder states in which NO levels are depressed;

hypertension;

disease or disorder of the immune system, such as diseases involving neutrophils or macrophages (e.g. atheromas) and leukocytes in the tonsils;

disease or disorder of the cardiac system;

disease or disorder of the liver;

disease or disorder of the pancreas;

disease or disorder of the nervous system, in particular any disease in which mutations of the connexins are implicated such as e.g. Charcot-Marie tooth disease and hereditary sensori-neural deafness;

disease or disorder of the lung vasculature or musculature such as for example asthma, where it is desired to assist a drug to cross the epithelium into the subjacent smooth muscle cells;

disease or disorder of the genito-urinary system, for example in kidney disease relating to tubular function;

the curative or prophylactic treatment of a neoplasm or tumour;

septic shock or other conditions involving hypotension;

disease or disorder of the immune system;

disease or disorder of the skin particularly diseases where enhanced drug penetration might be particularly efficacious, for example, antiviral agents for the treatment of shingles;

disease or disorder of the bronchial tree, particularly in the instance of viral pneumonia or in the case of asthma;

disease or disorder of blood vessels particularly, but not exclusively, the microcirculation and/or where one wishes to enable antiviral and anti neoplastic drugs to cross the vessel walls and enter tissues in high concentration. This strategy may be particularly useful in the case of neural tissue where the blood brain barrier often prevents drug access.

The synthetic peptide of the invention is preferably respectively homologous to one or more of the Gap 26 and 27 extracellular loop portions of a connexin protein.

In one aspect, the synthetic peptide may be VCYDQAFPISHIR, VCYDKSFPISHVR, SRPTEKTIFII or SRPTEKNVFIV.

Especially preferred synthetic peptides include RVDCFLSRPTEK, PVNCYVSRPTEK and IVDCYVSRPTEK, and in particular applications the synthetic peptide SRPTEKT may be used.

Where more than one synthetic peptide is used the combination, ideally, includes a triple peptide combination targeting connexin 37, 40 and 43. Using this combination we have found that in rat hepatic artery EDHF-type relaxations are unaffected by individual peptides, but abolished by the use of this triple peptide combination.

It is well-recognised that the importance of the EDHF phenomenon increases with diminishing vessel size and we have demonstrated that a two-fold larger EDHF response in small distal arteries, from the rabbit ear compared to the central artery, is specifically attributable to differences in gap junction or communication on the basis of experiments with connexin mimetic peptides. This tends to imply that direct endothelial-smooth muscle coupling may therefore be a particular functional importance in the micro circulation, consistent with morphological evidence that myoendothelial plaques are almost numerous in the distal vascular (25). Indeed, propagation of local responses longitudely along the vessel wall may also contribute to the coordinated function of the arteriolar network by integrating the intensity and nature of stimuli arriving from downstream sites. Gap junctions thus allow electrotonic propagation both of local dilations and the myogenic response. Indeed potentials generated in the endothelium spread with almost no reduction thus providing a functional correlate with a high incidence of interendothelial gap junction plaques evident on immunostaining or electron myoscroscopy. The endothelium may thus serve as the important low resistant path connecting multi smooth muscle cells as electrotonic potentials can conduct through myoenthelial gap junctions which behave as simple ohmic resistors without rectification.

According to a further aspect of the invention there is provided the use of cAMP or an adenylyl cylase activator or a cAMP PDE inhibitor, or a pharmaceutically acceptable derivative or salt thereof, in the production of a pharmaceutical composition effective for the curative or prophylactic treatment of a vascular disease.

In a preferred aspect of the invention said vascular disease is a disease of the microcirculation.

According to a further aspect of the invention there is provided the use of cAMP PDE inhibitor, or a pharmaceutically acceptable derivative or salt thereof, in the production of a pharmaceutical composition effective for the curative or prophylactic treatment of a vascular disease.

In a preferred aspect of the invention said vascular disease is a disease of the microcirculation.

According to a further aspect of the invention there is provided use of one or more synthetic peptides homologous to respective portions of one or more connexin proteins and effective to inhibit or attenuate intercellular gap junction communication, in the production of a pharmaceutical composition effective for the curative or prophylactic treatment of a vascular disease.

In a preferred aspect of the invention said vascular disease is of the microcirculation.

It should be noted that one of the advantages of treatment by the synthetic peptides is that they are non-permanent in the sense that after a certain period they are washed out and broken down or excreted from the blood circulation.

In another aspect, this invention provides a pharmaceutical composition for being administered within or on the body of an animal, including man, by causing said pharmaceutical composition to contact a surface within or on the body, said pharmaceutical composition comprising a therapeutic substance, or combination of such substances, in association with cAMP or an adenylyl cylase activator, or a cAMP PDE inhibitor, whereby on said composition contacting said surface, said cAMP or adenylyl cylase activator or cAMP PDE inhibitor initiates or enhances the transfer of said substance through said surface into subjacent cellular tissue, via one or more intercellular gap junctions.

In another aspect, this invention provides a pharmaceutical composition for being administered within or on the body of an animal, including man, by causing said pharmaceutical composition to contact a surface within or on the body, said pharmaceutical composition comprising a therapeutic substance, or combination of such substances, in association with a cAMP PDE inhibitor, whereby on said composition contacting said surface, said cAMP PDE inhibitor initiates or enhances the transfer of said substance through said surface into subjacent cellular tissue, via one or more intercellular gap junctions.

In a preferred aspect of the invention, said therapeutic substance comprises an antiviral agent. The antiviral agent may comprise any one or more of the following conventional viral agents or suitably modified analogues thereof: zidovudine, didanosine, zalcitabine, stavudine, lamivudine, ganciclovir, foscarnet, cidofovir, lodenosine, dideoxyadenosine, acyclovir.

Exemplified herein is the antiviral agent 2′,3′-dideoxyadenoine. This lipophilic antiviral agent is converted into a polar triphosphate once it has crossed the cell membrane and in its polar state it is able to penetrate into subcellular layers in its active form. More preferably, more lipophilic or hydrophobic derivatives of antiviral agents may be used. They are commonly known to those skilled in the art and some are described in detail in reference 32.

According to a yet further aspect this invention provides a pharmaceutical composition for being administered within or on the body of an animal, including man, by causing said pharmaceutical composition to contact a surface within or on the body, said pharmaceutical composition comprising a therapeutic substance linked or otherwise conjoined with a moiety designed to render the therapeutic substance permeant to the cell membrane, whereby on said composition contacting said surface, said moiety initiates or enhances the transfer of said therapeutic substance through the cell membrane into the cell, there to be cleaved from said substance to allow it to pass into subjacent cellular tissue via one or more intercellular gap junctions.

This aspect of the invention is visually illustrated by the dye transfer experiments described herein. We show in these experiments that the diffusion of the polar dye calcein from the endothelium to smooth muscle via gap junctions involves endothelium uptake of the lipophilic precursor calcein AM and intracellular cleavage by esterases to calcein, the polar form of the dye. Similarly, also illustrated herein, in the experiments using 2′,3′-DDA, is the conversion of the lipophilic agent to its active antiviral polar triphosphate, form via cellular enzymes, which would be able to diffuse through gap junctions in the same fashion as calcein.

In this aspect, the pharmaceutical composition may further include a cAMP or an adenylyl cylase activator a cAMP PDE inhibitor thereby further to assist transfer of said substance. Preferably, in this aspect, said therapeutic substance of said pharmaceutical composition is an antiviral agent. Ideally, said antiviral agent is any one or more of the following antiviral agents or a suitable derivative thereof: zidovudine, didanosine, zalcitabine, stavudine, lamivudine, ganciclovir, foscarnet, cidofovir, lodenosine, dideoxyadenosine, acyclovir.

Most preferably the antiviral agent is a lipophilic agent whose lipophilic moiety is cleaved once the agent has passed through the cell membrane leaving the polar component free in its active form to penetrate further into the tissue.

In the above aspects, the surface may comprise an endothelial region or an epithelial region. Thus typical examples include the skin, the arterial wall, the vascular system, and the blood/brain barrier. The epithelial region may be the lining of the lung, the colon or the bowel or may be the skin as identified or a mucus membrane.

In these aspects it is possible to use cAMP, and/or adenylyl cylclase activator and/or cAMP PDE inhibitor alone, or a synthetic connexin mimetic peptide alone, or these may be used together, either with or without the moiety described above, whereby the therapeutic substance or substances may be rendered permeant to the cell membrane. A further possible use of the synthetic peptides on the mucus membranes is to effect vasoconstriction for hay fever.

In another aspect, this invention provides a pharmaceutical composition comprising one or more of synthetic peptides targeted selectively to inhibit gap junction communication within the cells making up the blood vessels in selected regions or organs of the body of an animal including man, reversibly to inhibit relaxation thereof, thereby to cause enhanced blood flow elsewhere in the body.

In this way, a mixture of synthetic peptides may be administered specifically to enhance blood flow at a targeted site in the human or animal body. As discussed above, the synthetic peptides are non-permanent, being washed out and excreted or broken down to an inactive state after a short period.

The invention also extends to the following synthetic peptides:

VCYDQAFPISHIR
VCYDKSEPTISHVR
SRPTEKTIFIT
SRPTEKNVFIV
RVDCFLSRPTEK
PVNCYVSRPTEK
IVDCYVSRPTEK
SRPTEKT.

The invention also extends to methods or treatments of diseases, disorders or conditions using the cAMP, adenylyl cylase activator or cAMP PDE inhibitors or synthetic peptides as described above.

The invention also extends to a method of treating a condition which is responsive to modulation of Endothelium-derived hyperpoliarizing factor (EDHF) comprising administering to an individual to be treated a pharmaceutical composition comprising cAMP, an adenylyl cyclase activator or a cAMP phosphodiesterase inhibitor, or a pharmaceutically acceptable derivative or salt thereof, in combination with a selected therapeutic substance.

Further, the invention extends to a method of treating a condition which is responsive to modulation of Endothelium-derived hyperpoliarizing factor (EDHF) comprising administering to an individual to be treated a pharmaceutical composition comprising a cAMP phosphodiesterase inhibitor, or a pharmaceutically acceptable derivative or salt thereof, in combination with a selected therapeutic substance.

In a preferred method of the invention said condition is a disease or disorder of the vascular system or the immune system or the cardiac system or the liver or the pancreas or the nervous system or the respiratory system or the genito-urinary system or the skin or the brain or a neoplasm.

Whilst the invention has been described above, it extends to any inventive combination of the features set out in the remainder of the specification.

### DESCRIPTION

Agonists that act via the endothelium, such as acetylcholine (ACh), evoke smooth muscle hyperpolarizations and relaxations that are driven by a primary endothelial hyperpolarization and are independent of NO and prostanoid synthesis (11). Passive electrotonic mechanisms may contribute to the smooth muscle response as the endothelium and media are coupled electrically via myoendothelial gap junction plaques consisting of focal clusters of individual gap junctions constructed from connexin (Cx) proteins (6,25). Indeed, in arterioles endothelial hyperpolarization can be detected synchronously in smooth muscle, whether induced by ACh or the injection of electrical current into a single endothelial cell (12). By contrast, in thick-walled vessels it has been suggested that the endothelium cannot act as a major source of hyperpolarizing current because large differences in the mass of this monolayer and the media result in electrical mismatching (3). An alternative hypothesis, therefore, is that an endothelium-derived hyperpolarizing factor (EDHF) is released into the extracellular space to activate smooth muscle K⁺ channels and mediate relaxation (7,14,24).

There is nevertheless evidence that direct intercellular communication via gap junctions contributes to the EDHF phenomenon in conduit vessels. Synthetic peptides homologous to the Gap 26 or 27 domains of the 1^{st} and 2^{nd} extracellular loops of connexin proteins, which interrupt intercellular communication in a connexin-specific fashion, and 18α-glycyrrhetinic acid (18α-GA), a lipophilic aglycone that disrupts gap junction plaques, inhibit EDHF-type responses evoked by ACh in a spectrum of rabbit arteries and veins (4,8,10,15,17,26). Furthermore, in “sandwich” preparations of rabbit mesenteric artery, in which there can be no gap junctional communication between the endothelium of the donor tissue and smooth muscle of the detector tissue, relaxations evoked by ACh are mediated entirely by NO (8, 17). By contrast, sandwich experiments have provided evidence for the release of a factor, distinct from NO and prostanoids, that diffuses via the extracellular space following administration of the Ca²⁺ ionophore A23187 in rabbit femoral and mesenteric arteries (17,23). Furthermore, observations that EDHF-type relaxations evoked by ACh in the rabbit iliac artery are dependent on elevations in smooth muscle cAMP levels and phosphorylation events mediated by protein kinase A (PKA) nevertheless indicate that such responses may not simply be mediated by passive electrotonic mechanisms (27). Since cAMP accumulation is suppressed by interrupting gap junctional communication with connexin-mimetic peptides or 18α-GA, it is possible that chemical signalling contributes to the response to ACh, as in addition to providing electrical continuity, gap junctions allow direct transfer of signalling molecules <1 kDa in size between coupled cells. In the present study we demonstrate that cAMP similarly underpins the EDHF response to A23187, despite being independent of heterocellular communication, thereby providing evidence that similar biochemical events may underpin the EDHF phenomenon even when relaxation is effected via fundamentally different signalling pathways.

Materials and Methods

Isolated Ring Preparations. Male New Zealand white rabbits (2-2.5 kg) were sacrificed with sodium pentobarbitone (120 mg/kg; i.v.) and the iliac artery removed and transferred to cold Holman's buffer of the following composition (mM): 120 NaCl, 5 KCl, 2.5 CaCl₂, 1.3 NaH₂PO₄, 25 NaHCO₃, 11 glucose, and 10 sucrose. The vessels were stripped of adherent tissue, and rings 2-3 mm wide cut and suspended in organ chambers containing gassed (95% 02, 5% CO₂, pH 7.4) buffer at 37 C. Tension was set at 0.25 g and during an equilibrium period of 1 h the tissues were repeatedly washed with fresh buffer and tension readjusted following stress relaxation. Endothelium-intact rings were incubated for 40 min with N^{G}-nitro-L-arginine methyl ester (L-NAME, 300 μM) and indomethacin (10 μM) and following constriction with phenylephrine (PE) cumulative concentration-relaxation curves to ACh or A23187 were constructed. Some preparations were preincubated for 40 min with either 2′5′-dideoxyadenosine (2′5′-DDA, 200 μM), 3-isobutyl-1-methylxanthine (IBMX, 20 μM), 18α-GA (100 μM), glibenclamide (10 μM), the combination of charybdotoxin (100 nM) and apamin (300 nM), or the combination of charybdotoxin (100 nM), apamin (300 nM) and IBMX (20 μM). Concentration-response curves to ACh and A23187 were also constructed for endothelium-denuded rings in the absence/presence of IBMX (20 μM). In experiments with IBMX the concentration of PE used to induce tone was increased from 1 to 3 μM. All reagents were obtained from Sigma, U.K.

‘Sandwich’ preparations. Rings of iliac artery 2-3 mm wide were denuded of endothelium, cut into strips and pierced ˜2 mm from each end using a Monoject needle (0.9 mm×40 mm). These strips were introduced into the lumen of rings of endothelium-intact iliac artery 4-5 mm wide and the tissues sutured together. Composite preparations were then mounted in a Mulvany Multi Myograph (Danish Myo Technology) with the pierced denuded strips hooked onto the large vessel mountings. Tension was initially set at ˜0.25 g and readjusted during an equilibrium period of 1 h. The preparations were then incubated for 40 min with L-NAME (300 μM) and indomethacin (10 μM), constricted with PE (1 or 3 μM), and concentration-response curves constructed for ACh in the presence/absence of IBMX (20 μM), or A23187 in the presence/absence of IBMX (20 μM) or 2′5′DDA (200 μM).

Radioimmunoassay. Multiple rings from the same artery were incubated in oxygenated Holmans buffer containing L-NAME (300 μM) and indomethacin (10 μM) for 40 min at 37° C. in the presence or absence of 18α-GA (100 μM). Rings were frozen in liquid N₂ at time points up to 180 s following addition of ACh or A23187 and stored at −70° C. before extraction of cAMP or cGMP in 6% trichloroacetic acid, followed by neutralization with water saturated diethyl ether and subsequent radioimmunoassay (Amersham UK). PE (1 μM) was added 3 min before the initial control point and control experiments were performed with endothelium-denuded rings. Nucleotide levels were expressed relative to protein content (27).

Membrane Potential Experiments. Iliac artery strips were held adventitia down in an oxygenated (95% O₂, 5% CO₂) organ chamber by a Harp slice grid (ALA Scientific Instruments, USA) superfused (2 ml/min at 37° C.) with Holmans solution containing 300 μM L-NAME and 10 μM indomethacin. Transmembrane potential was recorded with glass capillary microelectrodes (tip resistance of 60-110 M□ filled with 3M KCl and connected to the headstage of a SEC-10LX amplifier (NPI Electronic, Germany). Successful impalements were achieved following a sudden negative drop in potential from baseline and a stable signal for at least 2 min. To ensure recordings were made from smooth muscle cells the microelectrode was advanced into the subintima using a PCS-5000 micromanipulator (Burleigh Instruments, UK) until there had been 2-3 such negative deflections. After first obtaining control responses to ACh by direct administration into the organ chamber under conditions of no flow, the strips were washed with fresh buffer before incubation with either 200 μM 2′,3′-DDA alone or the combination of 200 μM 2′,3′-DDA plus 30 μM IBMX followed by repeat exposure to ACh.

Dye Transfer. Femoral arteries were mounted in a Living Systems perfusion myograph (Living Systems Instrumentation, Burlington, Vt.) and perfused with oxygenated Holmans buffer (35° C.) at a flow rate of 0.1 ml/min at a constant pressure of 25 mmHg. The vessels were allowed to equilibrate for 30 min then perfused with 300 μM L-NAME and 10 μM indomethacin for 60 min followed, additionally, by either 600 μM Gap 27, 20 μM IBMX, 1 mM 8-bromo-cAMP, or 1 mM 8-bromo-cGMP for 30 min. The preparations were allowed to return to room temperature, and 10 μM calcein AM (Molecular Probes) was prefused through the lumen for 30 min before washout with dye-free buffer at 35° C. for 30 min. In control experiments arteries were perfused with 10 μM calcein, which is membrane impermeable. The vessels were subsequently removed from the myograph and fixed in 0.1 M PBS containing 0.2% glutaraldehyde and 2% formalin for 90 min before cryopreservation in OCT compound (Agar Scientific, Stanstead, UK), cooled by liquid N₂. Cryosections of transverse rings (10 μm thick) were prepared and mounted onto poly-L-lysine-coated slides under Fluorsave (Calbiochem) and imaged with a TCS four-dimensional confocal laser scanning system (Leica) with the filters set for 490 nm excitation and 525 nm emission. A gallery of several images was collected at 0.5-μm steps for each sample followed by image processing by using Leica SCANWARE software to obtain a maximum projection image. A pixel intensity profile across the vessel wall was then obtained with MATLAB software and fitted to a monoexponential to derive a space constant describing the decay of medial fluorescence as a function of distance from the intima, i.e., the distance over which fluorescence decremented by 1/e or ˜63%.

Statistical Analysis All data are given as mean±SEM, where n denotes the number of animals studied for each data point, and were compared by the Student's t-test for paired or unpaired data as appropriate. P<0.05 was considered as significant. Concentration-response curves were assessed by one-way analysis of variance (ANOVA) followed by the Bonferroni multiple comparisons test.

Results

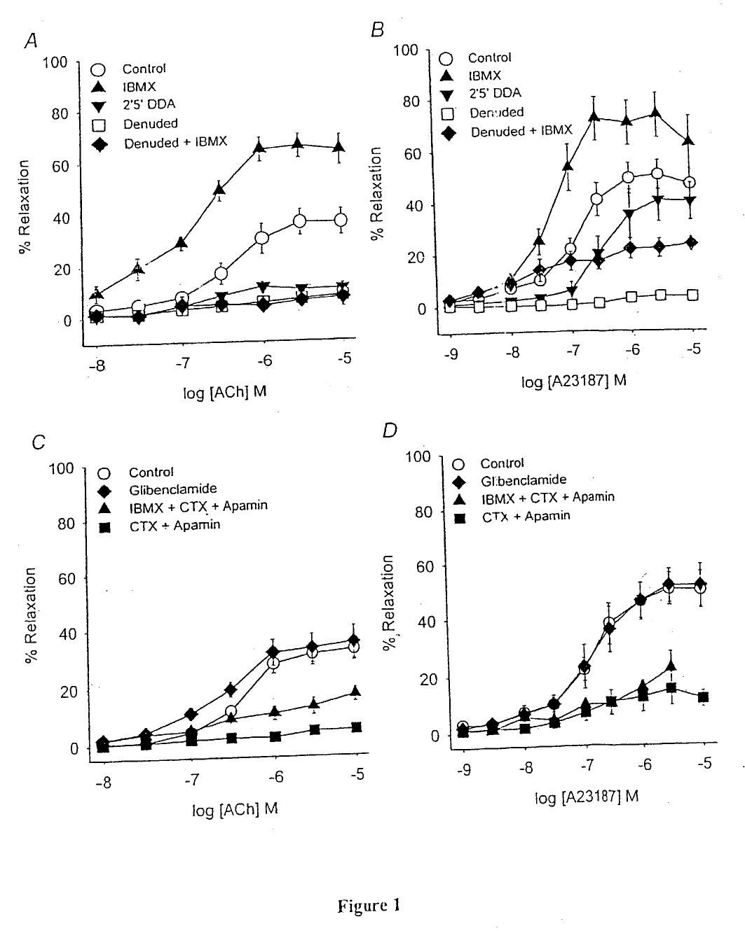

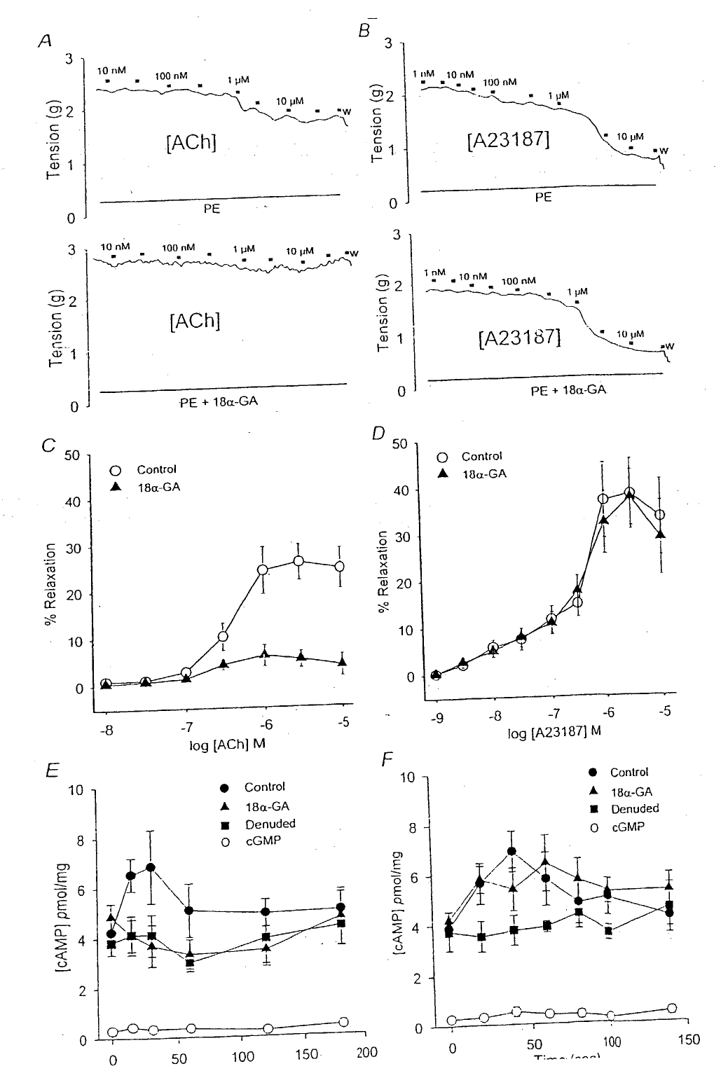

Isolated rabbit iliac artery rings. Maximal EDHF-type relaxations to ACh and A23187 were equivalent to ˜40% of PE-induced tone at concentrations ˜3 μM (FIGS. 1 and 2). In endothelium-intact rings IBMX (20 μM) potentiated these responses from 36.0±5.0% to 65.5±4.5% with ACh (P<0.05, n=14 and 10; FIG. 1A) and from 50.2±5.6% to 73.0+8.5% with A23187 (P<0.05, n=18 and 9; FIG. 1B), with corresponding leftward shifts in the EC₅₀ values from 555±121 nM to 119±21 nM and from 328±123 nM to 196±139 nM, respectively (P<0.05 in each case). Incubation with the adenyl cyclase inhibitor 2′,5′-DDA (200 μM) almost abolished ACh-evoked relaxations, with maximal responses reduced to 11.0±1.9% (P<0.05, n=4; FIG. 1A), whereas maximal relaxation to A23187 was reduced to 40.3±8.3% of PE-induced tone (P<0.05, n=9; FIG. 1B) with a shift in EC₅₀ value to 644±221 nM (P<0.05). Endothelial denudation abolished relaxations to both ACh and A23187 (n=8 and 5, respectively; FIGS. 1A and B). Incubation of endothelium-denuded rings with IBMX (20 μM) did not unmask relaxations to ACh (n=4; FIG. 1A), whereas A23187 induced a relaxation equivalent to 21.7±3.6% of PE-induced tone (P<0.05, n=6; FIG. 1B). The combination of charybdotoxin (100 nM) plus apamin (300 nM) abolished ACh-induced relaxations (P<0.05, n=5; FIG. 1C) and markedly attenuated responses to A23187 with maximal relaxation being reduced from 50.0±5.8% to 14.6±5.7% (P<0.05, n=3; FIG. 1D) of PE-induced tone, with a rightward shift in EC₅₀ values from 291±128 nM to 784±417 nM (P<0.05). The presence of IBMX (20 μM) reduced the effectiveness of the apamin plus charybdotoxin combination with maximal relaxations to ACh and A23187 reduced to 16.4±2.6% and 22.0±5.9% of PE-induced tone, respectively (n=4 and 3, respectively; FIGS. 1C and D). Incubation with glibenclamide (10 μM) did not significantly affect EDHF-type relaxations evoked by ACh or A23187 (n=5 and 9, respectively; FIGS. 1C and D).

In the presence of L-NAME (300 μM) and indomethacin (10 μM) 1 μM PE-evoked constrictions of 2.0±0.1 g (data pooled from all experiments with intact endothelium). Contractions were unaffected by endothelial denudation or incubation with 2′,5′-DDA (200 μM), glibenclamide (10 μM) or charybdotoxin (100 nM) plus apamin (300 nM). In experiments involving incubation with IBMX (20 μM) initial tension was restored to control levels by 3 μM PE (1.8±0.1 g, data pooled from all such experiments).

Effects of 18α-GA on relaxation and cAMP accumulation. The gap junction inhibitor 18α-GA (100 μM) effectively abolished EDHF-type relaxations evoked by ACh (n=5; FIG. 2 A and C), but had no effect on corresponding relaxations to A23187 (n=8; FIG. 2B and D). Similarly, 18α-GA (100 μM) abolished the transient rise in cAMP levels evoked by ACh (3 μM, n=5; FIG. 2E), but had no significant effect on nucleotide accumulation evoked by A23187 (3 μM, n=6; FIG. 2F). In endothelium-denuded preparations the basal level of cAMP was 3.97±0.49 pmol/mg protein and this did not significantly increase following exposure to either ACh or A23187 (n=4 in each case; FIG. 2E and F). In endothelial-intact preparations basal cAMP levels were 4.56±0.4 pmol/mg protein and did not differ significantly following incubation with L-NAME (300 μM) and indomethacin (10 μM) (n=9; FIG. 2E), whereas basal cGMP levels were reduced from 1.84±0.66 to 0.3±0.07 pmol/mg protein (P<0.05, n=3 and 8, respectively). Neither ACh nor A23187 significantly elevated cGMP levels in the presence of L-NAME and indomethacin (n=4 in each case; FIG. 2E and F).

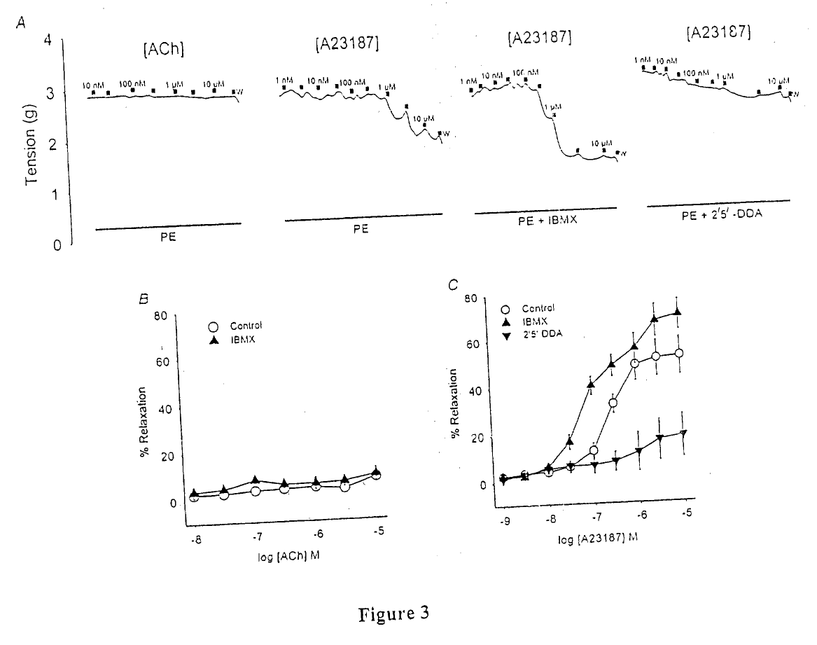

Sandwich preparations. ACh failed to evoke EDHF-type relaxations either in the presence or absence of IBMX (20 μM, n=5 in each case; FIG. 3A and B). By contrast, A23187 stimulated relaxations with a maximal response of 52.0±8.0% of PE-induced tone and an EC₅₀ value of 240±40 nM (n=5: FIG. 2A and C). Responses to A23187 were attenuated by 2′,5′-DDA (200 μM) with maximal relaxation reduced to 18.5±9.2% of PE-induced tone and a rightward shift in EC₅₀ to 1250±240 nM (P<0.05, n=5: FIG. 2A and C), and were potentiated by IBMX (20 μM) to a maximum of 70.0±6.50% with a leftward shift in EC₅₀ to 120±80 nM (P<0.05, n=5: FIG. 2A and C),

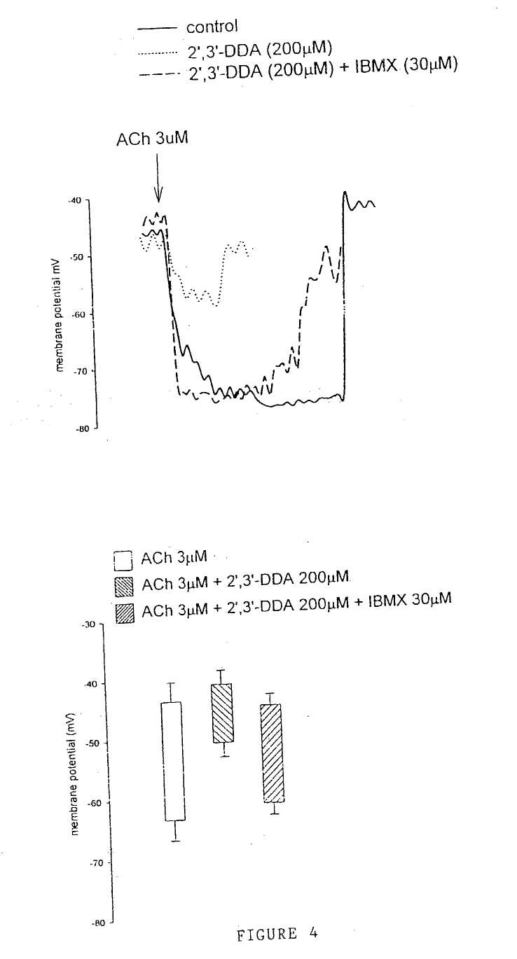

Membrane potential experiments. The role of cAMP in modulating myoendothekial gap junctions was investigated using sharp electrode electrophysiology. At a concentration of 200 μM the adenylyl cyclase inhibitor 2′, 3′-DDA attenuated transmission of electrical changes in endothelial membrane potential into subintimal smooth muscle cells by ˜75% (FIG. 4). This block was completely reversed by subsequent incubation with the cAMP phosphodiesterase inhibitor IBMX (30 μM).

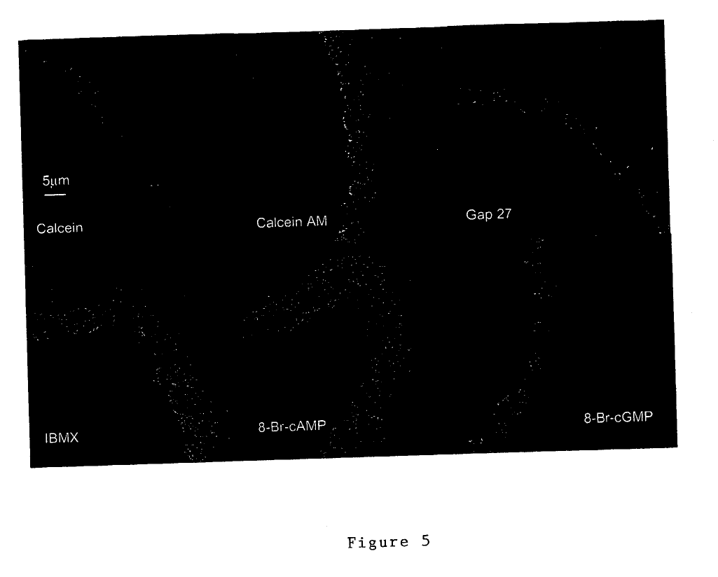

Dye Transfer. Dye was detected within the vessel wall after intraluminal perfusion with calcein AM but not calcein (FIG. 5). Under control conditions medial fluorescence decayed with a space constant of 8.54±0.39 μm (n=3), whereas dye was localized almost exclusively within the endothelium in arteries perfused with 600-μM Gap 27. Perfusion with 20 μM IBMX or 1 mM 8-bromo-cAMP significantly increased the space constant to 11.50±0.80 and 12.50±0.95 μm, respectively (n=6 and 5, P<0.05), whereas its value in arteries perfused with 1 mM 8-bromo-cGMP was 8.39+0.44 μm (n=4) and did not differ significantly from control.

Discussion

The present study has highlighted similarities and differences in the mechanisms that contribute to EDHF-type relaxations evoked by ACh and the Ca²⁺ ionophore A23187 in the rabbit iliac artery. The major finding is that the endothelium mediates NO- and prostanoid-independent relaxations to both agents by elevating smooth muscle cAMP levels, with the underlying signalling pathways involving myoendothelial gap junctions in the case of ACh but transfer of a diffusible factor via the extracellular space in the case of A23187.

In experiments with endothelium-intact rings, ACh and A23187 both evoked EDHF-type relaxations that were attenuated by inhibition of adenylate cyclase with the P site agonist 2′,5′-DDA and potentiated by inhibition of cAMP hydrolysis with IBMX. Administration of L-NAME significantly decreased basal cGMP levels and no subsequent elevations in levels of this nucleotide were detected following administration of ACh or A23187. This confirms near-maximal blockade of NO synthase and demonstrates that IBMX, which inhibits phosphodiesterases that hydrolyse both cGMP and cAMP (21), did not potentiate relaxation by amplifying the biochemical consequences of residual NO activity (9). Responses to ACh and A23187 were inhibited by the combination of apamin plus charybdotoxin, even when relaxation was potentiated by IBMX. This is a hallmark of the EDHF phenomenon and reflects the opening of apamin-sensitive small conductance channels (SK_{Ca}) and charybdotoxin-sensitive large and intermediate conductance channels (BK_{Ca} and IK_{Ca}) located on the endothelium (11). Experiments with glibenclamide excluded a role for cAMP-dependent activation of K_{ATP} channels in EDHF-type relaxations evoked either by ACh or A23187.

Confirmation that mechanical relaxations were dependent on elevations in smooth muscle cAMP levels was obtained by radioimmunoassay. In rings incubated with L-NAME and indomethacin, concentrations of ACh or A23187 resulting in similar maximal relaxations (˜40% of developed tone) were associated with endothelium-dependent 1.5 fold increases in cAMP levels, which are sufficient to elicit near-maximal biological responses (13). Although nucleotide levels returned to baseline within 3 minutes following application of either agent, elevations in cAMP were more sustained with A23187 (40-50s cf. 15-30s), consistent with the previously reported slower EDHF-type relaxation obtained with A23187 compared to ACh in rabbit mesenteric arteries (17). However, the signaling pathways activated by these agents were different, with 18α-GA abolishing ACh-induced relaxations and the associated cAMP accumulation, whereas the corresponding responses evoked by A23187 were unaffected. Analogous mechanical observations have been made in the rabbit superior mesenteric artery in which EDHF-type relaxations to ACh, but not A23187, are inhibited by connexin-mimetic peptides that interrupt gap junctional communication (17). The present findings with A23187 also indicate that 18α-GA does not inhibit cAMP synthesis non-specifically. Confirmation that the absolute cAMP content of the endothelial monolayer is small and contributes negligibly to nucleotide measurements in intact rings was provided by the observation that ACh did not elevate cAMP levels in endothelium-intact rings incubated with 18α-GA.

Bioassay experiments with sandwich preparations demonstrated the transfer of an endogenous vasodilator across the extracellular space following stimulation of the endothelium with A23187 under conditions of combined NO synthase and cyclooxygenase blockade. Observations that relaxations to A23187 were inhibited by 2′,5′-DDA and potentiated by IBMX confirm that cAMP mediates the associated mechanical response, as in intact rings. No transferable factor could be detected following administration of ACh, even in the presence of IBMX, which might have been expected to unmask the functional effects of subthreshold release of a freely diffusible mediator. Electrophysiological support for the hypothesis that A23187 promotes the extracellular release of an EDHF has also been provided by comparison of EDHF-type relaxations in the pig coronary artery evoked by A23187 and substance P (28). Mechanical relaxation and endothelium and smooth muscle hyperpolarizations evoked by substance P were inhibited by d-tubocurarine, which blocks SK_{Ca} channels, whereas the endothelial hyperpolarization evoked by 0.5 μM A23187 was abolished, but relaxation and smooth muscle hyperpolarization unaffected (28).

One explanation for these findings is that a chemical mediator, synthesized within the endothelium, transfers preferentially to smooth muscle via gap junctions following stimulation with ACh, whereas A23187 induces a large “overspill” of the same factor, thereby elevating smooth muscle cAMP levels via an extracellular route. Such a factor would also be expected to promote cAMP formation within the endothelium, and might contribute to the pronounced extracellular release of cAMP from the endothelium that is detectable in the effluent from buffer-perfused rabbit ear and rat mesentery preparations following administration of ACh or A23187 (1, 27). In the case of ACh, it is possible that diffusion of cAMP from the endothelium into the media via gap junctions contributes to the elevations in smooth muscle nucleotide levels, at least in part (15). The factor mediating relaxations to A23187 cannot, however, simply be cAMP derived from the endothelium as ACh-evoked efflux of this nucleotide does not modulate perfusion pressure in isolated rabbit ear preparations if gap junctional communication is interrupted by 18α-GA, presumably reflecting its low efficacy as an extracellular vasorelaxant (27). We have previously provided evidence that EDHF-type relaxations of rabbit arteries evoked by either ACh or A23187 require mobilization of arachidonic acid by a Ca²⁺-dependent phospholipase A₂ (17). In theory, this would be consistent with the hypothesis that epoxyeicosatrienoic acid (EET) metabolites of arachidonic acid function as freely diffusible EDHFs (7). Indeed, these compounds are synthesized by the endothelium, activate hyperpolarizing smooth muscle K⁺ channels, and elevate cAMP levels in cardiac myocytes and monocytes (7,29,30). However, in rabbit mesenteric arteries EDHF-type relaxations evoked by direct activation of phospholipase A₂ with the polypeptide melittin are mediated via gap junctions (18). Furthermore, 5,6-EET evokes relaxations that possess characteristics identical with ACh in that they are endothelium-, gap junction- and cAMP-dependent, and other EET regioisomers are inactive (17,27). These observations suggest that arachidonate metabolism within the endothelium may be an important initiating step in the EDHF phenomenon in rabbit arteries, but provide no support for the idea that the factor released by A23187 is an EET. The role of alternative hyperpolarizing arachidonate products such as the dihydroxyeicosatrienoic acids (DHET) in EDHF-type relaxations of rabbit arteries remains to be determined (20).

The central involvement of endothelial-smooth muscle coupling via gap junctions was confirmed by observations that relaxations and cAMP accumulation were both abolished by endothelial denudation and incubation with the connexin-mimetic peptide Gap 27. Because cAMP can enhance the molecular permeability and electrical conductance of gap junctions we investigated its ability to modulate dye transfer in the vessel wall. We selectively loaded the entire endothelium of femoral artery segments by intraluminal perfusion with the cell permeant dye calcein AM (31), so that fluorescence could be assessed in the media by confocal microscopy after hydrolysis to calcein. Transfer of calcein from the endothelium to subjacent smooth muscle cells was enhanced to an equivalent extent by IBMX and 8-bromo-cAMP, with the space constant for diffusion derived by quantitative image analysis increased from ≈8 to ≈12 μm in each case. Enhancement of dye transfer appeared specific for cAMP/8-bromo-cAMP because no analogous effect was observed after incubation with 8-bromo-cGMP. Although the role of myoendothelial gap junctions was confirmed by experiments with Gap 27, which markedly restricted penetration of calcein into the media, cAMP also modulates the subsequent diffusion of calcein via gap junctions coupling smooth muscle cells because there was substantially greater smooth muscle fluorescence in preparations incubated with IBMX or 8-bromo-cAMP.

To confirm the importance of cAMP-dependent modulation of myoendothelial gap junctions, we also investigated the effects of Ach on smooth muscle membrane potential in arterial strips impaled via their intimal surface. In preparations with intact endothelium 3 μM Ach evoked subintimal hyperpolarizations that were sustained ˜20 mV below baseline. The adenylyl cyclase inhibitor 2′,3′-DDA markedly attenuated these electrical changes and this inhibition was completely reversed by a low concentration of IBMX. Since electrical and chemical coupling of cells via gap junctions are in general equivalent, this observation indicates that 2′,3′-DDA will inhibit intercellular diffusion its own polar nucleoside triphosphate breakdown product, but that this effect will be revented by IBMX.

In conclusion, we have demonstrated that EDHF-type relaxations evoked by ACh and A23187 both depend on smooth muscle cAMP accumulation, but involve different intercellular communication pathways. Although the multiple cellular actions of cAMP encompass the diverse characteristics of the EDHF phenomenon reported in the literature, such as hyperpolarization via K_{Ca} channels and the Na⁺—K⁺ ATPase (11), it remains to be established if responses to ACh and A23187 involve a common chemical signal. Indeed, in the case of ACh there may be complex interactions between chemical and electrotonic signalling mechanisms as cAMP could in theory enhance relaxation by increasing the electrical conductance of gap junctions (2), thereby facilitating electrotonic spread of endothelial hyperpolarization into the media. Conducted endothelial hyperpolarization might also itself contribute to the smooth muscle cAMP accumulation evoked by ACh. EDHF-type relaxations are associated with closure of voltage-operated Ca²⁺ channels, resulting in a marked reduction in smooth muscle [Ca²⁺]ᵢ (5) that might activate the Ca²⁺-inhibited Type V and VI adenylyl cyclase isoforms that can be closely coupled to L-type Ca²⁺ channels and are expressed in vascular smooth muscle (19, 22). Alternatively, reductions in [Ca²⁺]ᵢ could suppress the Type I phosphodiesterase which is stimulated by Ca²⁺, thereby reducing cAMP hydrolysis and elevating cAMP levels (16).

### REFERENCES

1. Abiru T, Watanabe Y, Kamata K and Kasuya Y. Simultaneous measurement of vasodilation and changes in cyclic nucleotides in the perfused mesenteric arterial bed of the rat. Eur J Pharmacol 242: 15-22, 1993.

2. Abudara V, Eyzaguirre C and Saez, J C. Short- and long-term regulation of rat carotid body gap junctions by cAMP. Identification of connexin43, a gap junction subunit. Adv Exp Med Biol 475: 359-369, 2000.

3. Beny J L Information Networks in the Arterial Wall. News Physiol Sci 14: 68-73, 1999.

4. Berman R S, Martin P E M, Evans W H and Griffith T M. Relative contributions of NO and gap junctional communication to endothelium-dependent relaxations of rabbit resistance arteries vary with vessel size. Microvasc Res (63: 115-128,2002).

5. Bolz S S, De Wit C and Pohl U. Endothelium-derived hyperpolarizing factor but not NO reduces smooth muscle Ca²⁺ during acetylcholine-induced dilation of microvessels. Br J Pharmacol 128: 124-134, 1999.

6. Bukauskas F F, Jordan K, Bukauskiene A, Bennett M V, Lampe P D, Laird D W and Verselis V K. Clustering of connexin 43-enhanced green fluorescent protein gap junction channels and functional coupling in living cells. Proc Natl Acad Sci USA 97: 2556-2561, 2000.

7. Campbell W B and Harder D R. Endothelium-derived hyperpolarizing factors and vascular cytochrome P450 metabolites of arachidonic acid in the regulation of tone. Circ Res 84: 484-488, 1999.

8. Chaytor A T, Evans W H and Griffith T M. Central role of heterocellular gap junctional communication in endothelium-dependent relaxations of rabbit arteries. J Physiol(Lond) 508: 561-573, 1998.

9. Cohen R A, Plane F, Najibi S, Huk I, Malinski T and Garland C J. Nitric oxide is the mediator of both endothelium-dependent relaxation and hyperpolarization of the rabbit carotid artery. Proc Natl Acad Sci USA 94: 4193-8, 1997.

10. Dora K A, Martin P E M, Chaytor A T, Evans W H, Garland C J and Griffith T M. Role of heterocellular gap junctional communication in endothelium-dependent smooth muscle hyperpolarization: inhibition by a connexin mimetic peptide. Biochem Biophys Res Commun 254: 27-31, 1999.

11. Edwards G and Weston A H. EDHF-are there gaps in the pathway? J Physiol(Lond) 531: 299, 2001.

12. Emerson G G and Segal S S. Endothelial cell pathway for conduction of hyperpolarization and vasodilation along hamster fee artery. Circ Res 86: 94-100,2000.

13. Francis S H and Corbin J D. Structure and function of cyclic nucleotide-dependent protein kinases. Ann Rev Physiol 56: 237-272, 1994.

14. Gebremedhin D, Harder D R, Pratt P F and Campbell W B. Bioassay of an endothelium-derived hyperpolarizing factor from bovine coronary arteries: role of a cytochrome P450 metabolite. J Vasc Res 35: 274-84, 1998.

15. Griffith T M and Taylor H J. Cyclic AMP mediates EDHF-Type relaxations of rabbit jugular vein. Biochem Biophys Res Comm 263: 52-57, 1999.

16. Houslay M D. Adaptation in cyclic AMP signalling processes: a central role for cyclic AMP phosphodiesterases. Semin Cell Dev Biol 9: 161-7, 1998.

17. Hutcheson I R, Chaytor A T, Evans W H and Griffith T M. Nitric oxide-independent relaxations to acetylcholine and A23187 involve different routes of heterocellular communication. Role of gap junctions and phospholipase A2. Circ Res 84: 53-63, 1999.

18. Hutcheson I R and Griffith T M. Role of phospholipase A(2) and myoendothelial gap junctions in melittin-induced arterial relaxation. Eur J Pharmacol 406: 239-45, 2000.

19. Jourdan K B, Mason N A, Long L, Philips P G, Wilkins M R and Morrell N W. Characterization of adenylyl cyclase isoforms in rat peripheral pulmonary arteries. Am J Physiol 280: L1359-L1369, 2001.

20. Lu T, Katakam P V, VanRollins M, Weintraub N L, Spector A A and Lee H C. Dihydroxyeicosatrienoic acids are potent activators of Ca(2+)-activated K(+) channels in isolated rat coronary arterial myocytes. J Physiol(Lond) 534: 651-67, 2001.

21. Lugnier C and Komas N. Modulation of vascular cyclic nucleotide phosphodiesterases by cyclic GMP: role in vasodilatation. Eur Heart J 14 Suppl I: 141-8, 1993.

22. Murthy K S and Makhlouf G M. Regulation of adenylyl cyclase type V/VI in smooth muscle: interplay of inhibitory G protein and Ca²⁺ influx. Mol Pharmacol 54: 122-128, 1998.

23. Plane F, Pearson T and Garland C J. Multiple pathways underlying endothelium-dependent relaxation in the rabbit isolated femoral artery. Br J Pharmacol 115: 31-8, 1995.

24. Popp R, Bauersachs J, Hecker M, Fleming I and Busse R. A transferable, beta-naphthoflavone-inducible, hyperpolarizing factor is synthesized by native and cultured porcine coronary endothelial cells. J Physiol(Lond) 497: 699-709, 1996.

25. Sandow S L and Hill C E. Incidence of myoendothelial gap junctions in the proximal and distal mesenteric arteries of the rat is suggestive of a role in endothelium-derived hyperpolarizing factor-mediated responses. Circ Res 86: 341-346, 2000.

26. Taylor H J, Chaytor A T, Evans W H and Griffith T M. Inhibition of the gap junctional component of endothelium-dependent relaxations in rabbit iliac artery by 18-α-glycyrrhetinic acid. Br J Pharmacol 125: 1-4, 1998.

27. Taylor H J, Chaytor A T, Edwards D H and Griffith T M. Gap junction-dependent increases in smooth muscle cAMP underpin the EDHF phenomenon in rabbit arteries. Biochem Biophys Res Commun 283: 583-9, 2001.

28. Von der Weid P Y and Beny J L. Effect of Ca2+ ionophores on membrane potential of pig coronary artery endothelial cells. Am J Physiol 262: H1823-H1831, 1992.

29. Wong P Y, Lai P S and Falck J R. Mechanism and signal transduction of 14 (R), 15 (S)-epoxyeicosatrienoic acid (14,15-EET) binding in guinea pig monocytes. Prostaglandins Other Lipid Mediat 62: 321-333, 2000.

30. Xiao Y F, Huang L and Morgan J P. Cytochrome P450: a novel system modulating Ca2+ channels and contraction in mammalian heart cells. J Physiol(Lond) 508: 777-92, 1998.

31. Goldberg, G S, Bechberger, J F & Naus C C. (1995)BioTechniques 18, 490-497.

32. Siddiqui, McGuigan, Ballatore, Wedgewood, De Clerq, Balzarini. Bioorg Med Chem Lett. 9; 2555-2560, 1999.

33. Chaytor, Martin, Edwards, Grifith. Gap junctional communication underpins EDHF-type relaxations evoked by Ach in the rat hepatic artery Am. J. Physiol 280:H2441-2450, 2001.

Figure Legends

FIG. 1. Concentration-response curves showing EDHF-type relaxations evoked by ACh (A and C) and A23187 (B and D). Relaxations to both agents were potentiated by IBMX (20 μM, n=10 and 9, respectively; A and B) and abolished by endothelial denudation (n=8 and 5, respectively; A and B). ACh-evoked relaxations were essentially abolished by 2′5′-DDA (200 μM) and responses to A23187 attenuated (n=4 and 9, respectively; A and B). No relaxation to ACh was evident in endothelium-denuded rings incubated with IBMX (20 μM) whereas A23187 induced a small relaxation (n=4 and 6, respectively; A and B). Preincubation with glibenclamide (10 μM) did not affect relaxations evoked by ACh or A23187 (n=5 and 9, respectively; C and D). The combination of charybdotoxin (100 nM) plus apamin (300 nM) virtually abolished control relaxations (n=5 and 3; C and D) and markedly attenuated the potentiating effects of IBMX (n=4 and 3, respectively; C and D).

FIG. 2. Representative traces (A and B) and concentration-response curves (C and D) showing abolition of EDHF-type ACh-evoked relaxations by 18α-GA (100 μM), but no effect on corresponding responses to A23187 (n=5 and 8, respectively). In endothelium-intact rings incubated with L-NAME (300 μM) and indomethacin (10 μM), ACh (3 μM) evoked a transient peak in cAMP levels at 15-30 sec (n=4; E) which was abolished by 18α-GA (100 μM, n=6), 2′,5′-DDA (200 μM, n=5) and endothelial denudation (n=5). Accumulation of cAMP evoked by A23187 (3 μM) peaked at 40-50 sec (n=6; F) and was unaffected by 18α-GA (100 μM, n=6) but abolished by endothelial denudation (n=5). cGMP levels were unaltered by ACh or A23187 (E and F).

FIG. 3. Representative traces (A) and concentration-response curves (B and C) in sandwich preparations incubated with L-NAME (300 μM) and indomethacin (10 μM). ACh failed to induce relaxations either in the presence or absence of IBMX (20 μM, n=5 in each case; A and B). By contrast, A23187 evoked relaxations that were attenuated by 2′,5′-DDA (200 μM) and potentiated by IBMX (n=5 in each case; A and C).

FIG. 4. Effects of 3 μM acetylcholine on subintimal smooth muscle membrane potential in endothelium-intact strips of rabbit iliac artery. (a) Representative traces showing rapid initial hyperpolarizations of Ach that were sustained 20 MV below resting level. Changes in membrane potential were markedly attenuated by 200 μM 2′,3′-DDA but completely restored by 30 μM IBMX. (b) Histograms show results pooled from 5 such experiments. *P<0.05 compared to control response.

FIG. 5. Dye transfer in isolated rabbit femoral areteries. After intraluminal perfusion with calcein, only autofluorescence of the internal elastic lamina was evident, whereas the cell permeant calcein AM allowed penetration of dye into subjacent smooth muscle cells. Diffusion of dye was prevented by 600 μM Gap 27, enhanced by 20 μM IBMX and 1 mM 8-bromo-cAMP, but unaffected by 1 mM 8-bromo-cGMP. All images shown at the same magnification.

Detailed Structure of the Gap Junction and the Connexins

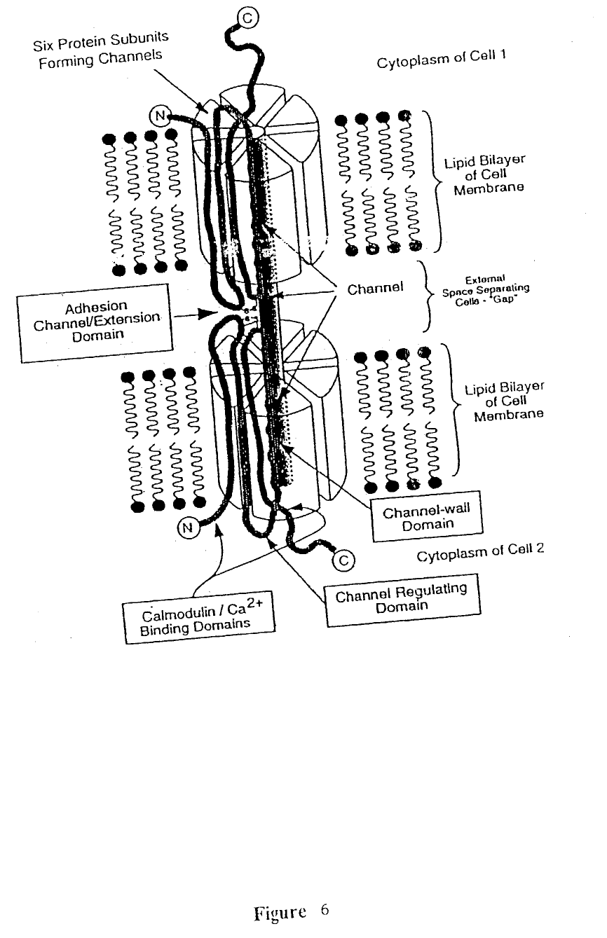

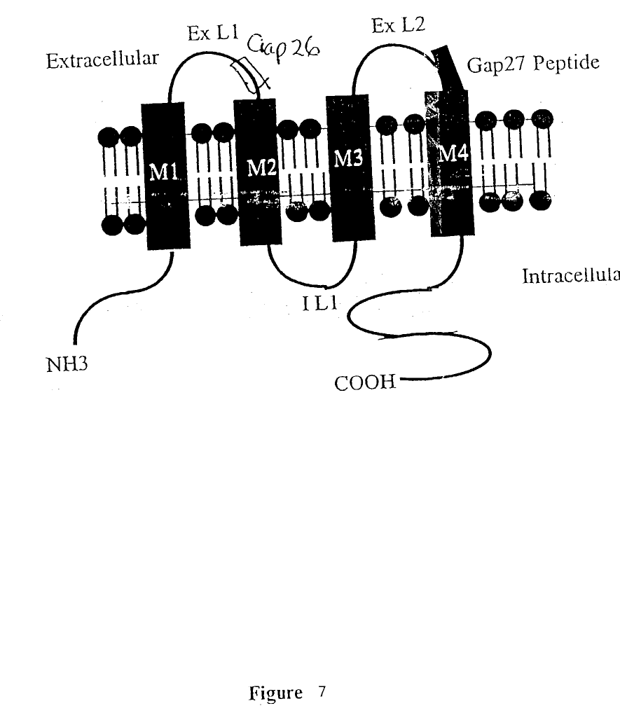

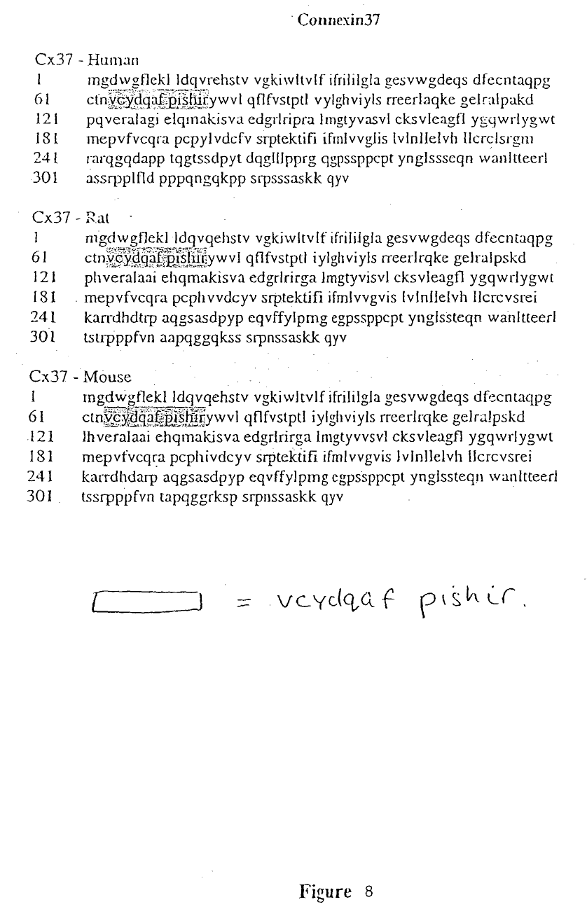

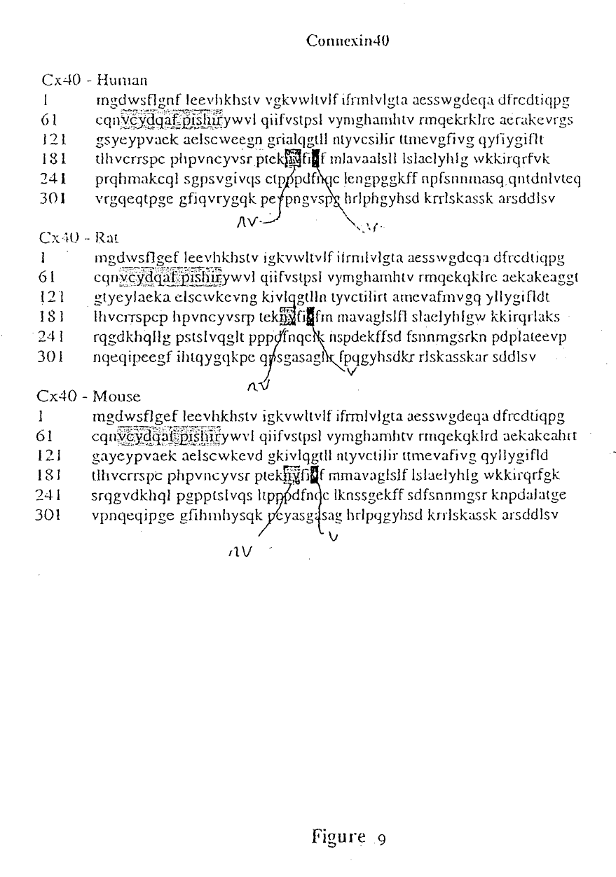

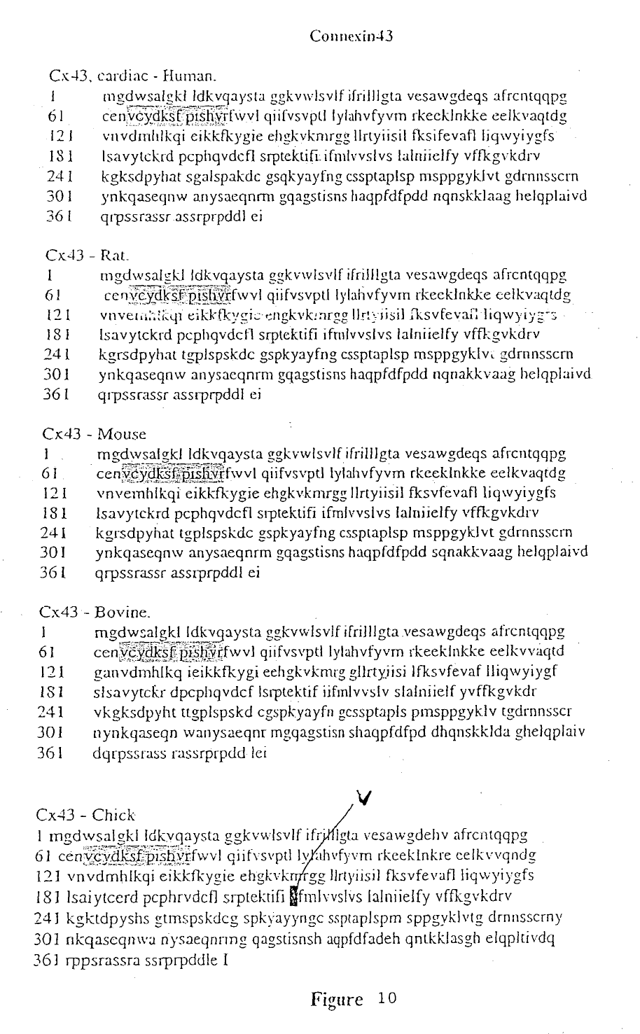

The detailed structure of the gap junction and that of the connexins is illustrated in FIGS. 6 and 7. The amino acid sequence listings for connexins 37, 40 and 43 are given in FIGS. 8, 9 and 10 for human and other species as indicated in the relevant Figures.

1. A pharmaceutical composition for being administered within or on the body of an animal, including man, by causing said pharmaceutical composition to contact a surface within or on the body, said pharmaceutical composition comprising a therapeutic substance, or a combination of such substances, in association with cAMP, or adenylyl cyclase activator or cAMP phosphodiesterase inhibitor, or a pharmaceutically acceptable derivative or salt thereof, whereby on said composition contacting said surface, said cAMP or adenylyl cyclase activator or cAMP phosphodiesterase inhibitor initiates or enhances the transfer of said substance through said surface into subjacent cellular tissue via one or more intercellular gap junctions.

2. A pharmaceutical composition according to claim 1 wherein said adenylyl cyclase activator is exogenous or synthetic.

3. A pharmaceutical composition according to claim 2 wherein said adenylyl cyclase activator is salbutamol.

4. A pharmaceutical composition according to claim 1 wherein said cAMP phosphodiesterase inhibitor is exogenous or synthetic.

5. A pharmaceutical composition according to claim 4 wherein said inhibitor is any one or more of the following inhibitors: IBMX, Rolipram or Milrinone.

6. A pharmaceutical composition according to claim 1 wherein said cAMP is exogenous or synthetic.

7. A pharmaceutical composition according to claim 1 wherein said pharmaceutical composition comprises an antiviral agent.

8. A pharmaceutical composition according to claim 7 wherein said antiviral agent is lipophilic or hydrophobic.

9. A pharmaceutical composition according to claim 7 or 8 wherein said agent is any or more of the following agents: dideoxyadenosine, zidovudine, didanosine, zalcitabine, stavudine, lamivudine, ganciclovir, foscarnet, cidofovir, lodenosine, acyclovir.

10. A pharmaceutical composition according to claim 1 wherein said condition comprises a disease or disorder of the vascular system or the immune system or the cardiac system or the liver or the pancreas or the nervous system or the respiratory system or the genito-urinary system or the skin or the brain or a neoplasm.

11. A pharmaceutical composition according to claim 10 wherein said condition is a disease of the microvascular system.

12. The use of cAMP, an adenylyl cyclase activator or a cAMP phosphodiesterase inhibitor, or a pharmaceutically acceptable derivative or salt thereof, in the production of a pharmaceutical composition effective for the curative or prophylactic treatment of a condition, disease or disorder in at animal, including man, which is responsive to modulation of Endothelium-derived hyperpolarizing factor (EDHF).

13. Use according to claim 12 wherein said adenylyl cyclase activator is exogenous or synthetic.

14. Use according to claim 13 wherein said adenylyl cyclase activator is salbutamol.

15. Use according to any claim 12 wherein said cAMP phosphodiesterase inhibitor is exogenous or synthetic.

16. Use according to claim 15 wherein said inhibitor is any one or more of the following inhibitors: IBMX, Rolipram or Milrinone.

17. Use according to claim 12 wherein said cAMP is exogenous or synthetic.

18. Use according to claim 12 wherein said pharmaceutical composition comprises an antiviral agent.

19. Use according to claim 18 wherein said antiviral agent is lipophilic or hydrophobic.

20. Use according to claims 18 or 19 wherein said agent is any or more of the following agents: dideoxyadenosine, zidovudine, didanosine, zalcitabine, stavudine, lamivudine, ganciclovir, foscarnet, cidofovir, lodenosine, acyclovir.

21. Use according to claim 12, wherein said condition comprises a disease or disorder of the vascular system or the immune system or the cardiac system or the liver or the pancreas or the nervous system or the respiratory system or the genito-urinary system or the skin or the brain or a neoplasm.

22. Use according to claim 21 wherein said condition is a disease of the microvascular system.

23. A pharmaceutical composition for being administered within or on the body of an animal, including man, by causing said pharmaceutical composition to contact a surface within or on the body, said pharmaceutical composition comprising a therapeutic substance linked or otherwise cojoined with a moiety designed to render the therapeutic substance permeant to the cell membrane, whereby on said composition contacting said surface said moiety initiates or enhances the transfer of said therapeutic substance through the cell membrane into the cell, there to be cleaved from said substance to allow it to pass into subjacent cellular tissues via one or more intercellular gap junctions.

24. The use of one or more synthetic peptides homologous to respective portions of one or more connexin proteins and effective to inhibit or attenuate intercellular gap junction communication, in the production of a pharmaceutical composition effective for the curative or prophylactic treatment of a condition, disease or disorder in an animal, including man, that is responsive to inhibition or attenuation of intercellular gap junction communication.

25. Use according to claim 24 wherein said synthetic peptidue(s) comprises any one or more of the following peptides.
VCYDQAFPISHIR
VCYDKSFPISHVR
SRPTEKTIFII
SRPTEKNVFIV
RVDCFLSRPTEK
PVNCYVSRPTEK
IVDCYVSRPTEK
SRPTEKT.

26. A pharmaceutical composition comprising one or more synthetic peptides targeted selectively to inhibit gap junction communication within the cells making up the blood vessels in selected regions or organs of the body of an animal, including man, reversibly to inhibit relaxation thereof, thereby causing enhanced bloodflow elsewhere in the body.

27. A pharmaceutical composition according to claim 26 wherein said synthetic peptidue(s) comprises any one or more of the following peptides.
VCYDQAFPISHTR
VCYDKSFPISHVR
SRPTEKTIFII
SRPTEKNVFIV
RVDCFLSRPTEK
PVNCYVSRPTEK
IVDCYVSRPTEK
SRPTEKT.
